(19) United States
(12) Patent Application Publication
(10) Pub. No.: US 2025/0257654 A1
(43) Pub. Date: Aug. 14, 2025

(54) DESIGN OF BOREHOLE COMPLETION PLAN BASED ON A FAILURE MODE OF ROCK

(71) Applicant: ARAMCO SERVICES COMPANY, Houston, TX (US)

(72) Inventor: Yanhui Han, Houston, TX (US)

(73) Assignee: ARAMCO SERVICES COMPANY, Houston, TX (US)

(21) Appl. No.: 18/441,793

(22) Filed: Feb. 14, 2024

(51) Int. Cl. E21B 49/00; E21B 49/02; G01N 33/24

(52) U.S. Cl. CPC E21B 49/006; E21B 49/02; G01N 33/24; E21B 2200/20

(57) ABSTRACT

Methods and systems are disclosed. Methods may include obtaining, from a rock characterization system, rock data for a rock that surrounds an interval of a borehole within a formation and determining principal in-situ effective stresses and mechanical properties of the rock using the rock data. The methods may further include determining, using a first model, a breakdown pressure for the rock using the principal in-situ effective stresses and the mechanical properties and determining, using a second model, a yield pressure for the rock using the principal in-situ effective stresses and the mechanical properties. The methods may still further include determining a deformation mechanism and a failure mode of the rock by comparing the breakdown pressure and the yield pressure and designing, using a completion design system, a completion plan for the borehole based on the deformation mechanism, the failure mode, and the breakdown pressure.

### BACKGROUND

Hydrocarbons may be stored in-situ within rock that forms an unconventional hydrocarbon reservoir. A hydraulic fracturing operation may be performed to induce fractures within the rock such that the stored hydrocarbons may be produced from the rock to the surface of the earth via a borehole. Typically, the hydraulic fracturing operation raises the pressure of a borehole fluid above a breakdown pressure to fracture the rock. However, the rock may fail to fracture above the breakdown pressure. Failure to fracture results in the loss of time and money. Further, failure to fracture results in the loss of production as hydrocarbons stored within the rock may not be produced from the rock unless fractured due to the low permeability of the rock.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method. The method includes obtaining, from a rock characterization system, rock data for a rock that surrounds an interval of a borehole within a formation and determining principal in-situ effective stresses and mechanical properties of the rock using, at least in part, the rock data. The method further includes determining, using a first model, a breakdown pressure for the rock using, at least in part, the principal in-situ effective stresses and the mechanical properties and determining, using a second model, a yield pressure for the rock using, at least in part, the principal in-situ effective stresses and the mechanical properties. The method further still includes determining a deformation mechanism and a failure mode of the rock by comparing the breakdown pressure and the yield pressure and designing, using a completion design system, a completion plan for the borehole based, at least in part, on the deformation mechanism, the failure mode, and the breakdown pressure.

In general, in one aspect, embodiments relate to a system. The system includes a computer system and a completion design system. The computer system is configured to receive, from a rock characterization system, rock data for a rock that surrounds an interval of a borehole within a formation and determine principal in-situ effective stresses and mechanical properties of the rock using, at least in part, the rock data. The computer system is further configured to determine, using a first model, a breakdown pressure for the rock using, at least in part, the principal in-situ effective stresses and the mechanical properties and determine, using a second model, a yield pressure for the rock using, at least in part, the principal in-situ effective stresses and the mechanical properties. The computer system is still further configured to determine a deformation mechanism and a failure mode of the rock by comparing the breakdown pressure and the yield pressure. The completion design system is configured to design a completion plan for the borehole based, at least in part, on the deformation mechanism, the failure mode, and the breakdown pressure.

In general, in one aspect, embodiments relate to a non-transitory computer-readable memory having computer-executable instructions stored thereon that, when executed by a computer processor, perform steps. The steps include receiving, from a rock characterization system, rock data for a rock that surrounds an interval of a borehole within a formation and determining principal in-situ effective stresses and mechanical properties of the rock using, at least in part, the rock data. The steps further include determining, using a first model, a breakdown pressure for the rock using, at least in part, the principal in-situ effective stresses and the mechanical properties and determining, using a second model, a yield pressure for the rock using, at least in part, the principal in-situ effective stresses and the mechanical properties. The steps further still include determining a deformation mechanism and a failure mode of the rock by comparing the breakdown pressure and the yield pressure and designing, using a completion design system, a completion plan for the borehole based, at least in part, on the deformation mechanism, the failure mode, and the breakdown pressure.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 illustrates a cross section of a formation in accordance with one or more embodiments.

FIG. 2 displays a pressure-displacement curve in accordance with one or more embodiments.

FIG. 3 describes a method in accordance with one or more embodiments.

FIG. 4 illustrates a computer system in accordance with one or more embodiments.

FIG. 5 illustrates a hydraulic fracturing operation in accordance with one or more embodiments.

FIG. 6 illustrates a flowchart of systems in accordance with one or more embodiments.

FIG. 7 displays an example pressure-displacement curve in accordance with one or more embodiments.

### DETAILED DESCRIPTION

In the following detailed description of embodiments of the disclosure, numerous specific details are set forth to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before,” “after,” “single,” and other such terminology. Rather, the use of ordinal numbers is to distinguish between the elements. By way of an example, a first element is distinct from a second element, and the first element may encompass more than one element and succeed (or precede) the second element in an ordering of elements.

It is to be understood that the singular forms “a,” “an,” and “the” include plural referents unless the context clearly dictates otherwise. Thus, for example, reference to “a principal in-situ effective stress” includes reference to one or more of such stresses.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

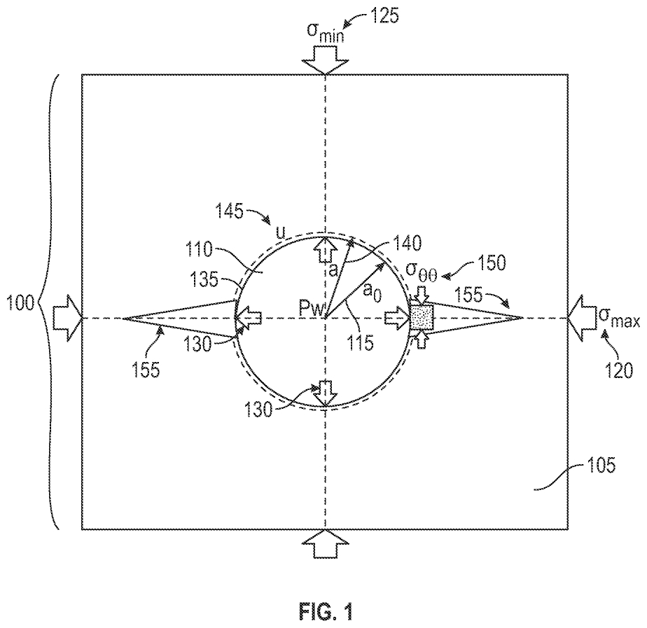

In the following description of FIGS. 1-7, any component described regarding a figure, in various embodiments disclosed herein, may be equivalent to one or more like-named components described regarding any other figure. For brevity, descriptions of these components will not be repeated regarding each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described regarding a corresponding like-named component in any other figure.

Methods and systems are disclosed to design a completion plan for a borehole. The term “completion” may describe the systems and/or operations used to bring the borehole into production or return the borehole to production. During production, hydrocarbons are produced from a hydrocarbon reservoir to the surface of the earth via the borehole that penetrates the hydrocarbon reservoir.

The term “openhole” may refer to the drilled-out portion of a formation that is uncased (i.e., casing is not installed downhole). The terms “borehole,” “wellbore,” and “well,” which are often used interchangeably, may not indicate if casing is installed downhole. Hereinafter, the term “borehole” is adopted. The borehole may be completely drilled or partially drilled. The borehole may be uncased, partially cased, or cased. The borehole may or may not penetrate a hydrocarbon reservoir within the formation.

The hydrocarbon reservoir may be an unconventional hydrocarbon reservoir. The unconventional hydrocarbon reservoir may be made up of layers of rock, such as shale or mudstone, that store hydrocarbons within pores of the rock. Permeability is the measure of how easily fluids, such as the hydrocarbons, flow through the rock. Unconventional hydrocarbon reservoirs typically have extremely low permeability. As such, the hydrocarbons may be stationary or slowly flow through the rock.

Turning to FIG. 1, FIG. 1 illustrates a cross section of a formation 100 in accordance with one or more embodiments. The cross section intersects rock 105 within the formation 100. Further, the cross-section intersects an interval of a vertical borehole 110 previously drilled within the rock 105. The borehole 110 is initially drilled to an initial radius a₀ 115.

The rock 105 may be characterized by mechanical properties. The mechanical properties may include, but are not limited to friction angle, dilation angle, Biot's coefficient, Poisson's ratio, tensile strength, unconfined compressive strength, shear modulus, Young's modulus, and cohesion strength. Some mechanical properties may be specifically referred to as elastic properties and/or poroelastic properties. For example, Young's modulus may be an elastic property and Biot's coefficient may be a poroelastic property. Some mechanical properties are dependent on other mechanical properties. For example, the unconfined compressive strength may be dependent on cohesion strength and friction angle.

Further, the rock 105 may be subjected to principal in-situ effective stresses and pore pressure. The principal in-situ effective stresses confine the rock 105 and, as such, may be written in terms of confining stress. When represented in a cylindrical coordinate system, the principal in-situ effective stresses around the borehole 110 may be written in terms of radial stress and/or tangential stress.

Turning to the principal in-situ effective stresses, local rock 105 may experience three principal in-situ effective stresses each of which are orthogonal to a surface or face of the local rock 105, where the local rock 105 may take any shape. FIG. 1 illustrates two horizontal principal in-situ effective stresses: a maximum horizontal principal in-situ effective stress σₘₐₓ 120 and minimum horizontal principal in-situ effective stress σₘᵢₙ 125. A third principal in-situ effective stress (not shown) exists normal to the maximum horizontal principal in-situ effective stress σₘₐₓ 120 and minimum horizontal principal in-situ effective stress σₘᵢₙ 125. The third principal in-situ effective stress may also be referred to as a vertical principal in-situ effective stress or overburden principal in-situ effective stress. The principal in-situ effective stresses may be compressive, anisotropic, and nonhomogeneous. Hereinafter, two or more of the maximum horizontal principal in-situ effective stress σₘₐₓ 120, minimum horizontal principal in-situ effective stress σₘᵢₙ 125, and vertical principal in-situ effective stress are generically referred to as “principal in-situ effective stresses.”

The magnitude of each principal in-situ effective stress may be controlled by other stresses and/or pressures. The other stresses and/or pressures may include, but are not limited to, the pore pressure Pₚ, surrounding stress, overburden stress, and injection pressure P_{w} 130. The pore pressure Pₚ may be caused by fluid (e.g., the hydrocarbons) stored within the pores of the rock 105. The surrounding stress and overburden stress may be caused by surrounding and overburden rock. The injection pressure P_{w} 130 may be caused by fluid injected into the rock 105 via the borehole 110. The injection pressure P_{w} 130 may place stress on the walls 135 of the borehole 110 as illustrated in FIG. 1.

One or more of these other stresses and/or pressures may, in turn, cause the initial radius a₀ 115 of the borehole 110 to expand to expanded radius a 140. The difference between the initial radius a₀ 115 and the expanded radius a 140 may be denoted radial displacement u 145.

If the rock 105 is assumed to elastically deform, the maximum horizontal principal in-situ effective stress σₘₐₓ 120, minimum horizontal principal in-situ effective stress σₘᵢₙ 125, and injection pressure P_{w} 130 may control a minimum tangential stress σ_{θθ} 150 located at the walls 135 of the borehole 110 based on the Kirsch solution where:

$${σ}_{θθ} = 3 {σ}_{min} - {σ}_{max} - {P}_{w} . \qquad Equation ( 1 )$$

The maximum horizontal principal in-situ effective stress σₘₐₓ 120 and minimum horizontal principal in-situ effective stress σₘᵢₙ 125 along with the tensile strength T of the rock 105 may control, at least in part, the pressure required to generate a fracture 155 within the rock 105 (i.e., fail the rock 105). Hereinafter, the pressure required to generate a fracture 155 within the rock 105 is denoted breakdown pressure P_{b}. Further, hereinafter, the failure mode of the rock 105 following elastic deformation is denoted brittle fracture. The fracture 155 may propagate perpendicular to the minimum horizontal principal in situ stress σₘᵢₙ 125 as illustrated in FIG. 1. For example, if fluid is injected into the rock 105 via the borehole 110 at high pressures, the minimum tangential stress σ_{74 θ} may become tensile and increase above the tensile strength T of the rock 105 and cause the rock 105 to fracture. The fracture 155 may occur at or near the breakdown pressure P_{b} where:

$${P}_{b} = 3 {σ}_{min} - {σ}_{max} + T . \qquad Equation ( 2 )$$

Equation (2) is proposed by Hubbert and Willis and referred to as the H-W model. In the context of this disclosure, Equation (2) may be a first model or a modified version of Equation (2) may be the first model. For example, the breakdown pressure P_{b} may be considered a relative pressure above the pore pressure Pₚ and, as such, the true breakdown pressure P_{b} may be P_{b}=3 σₘᵢₙ−σₘₐₓ+T+Pₚ.

However, Equation (2) assumes the rock 105 elastically deforms and that the borehole 110 is impermeable. If the borehole 110 is assumed to elastically deform and be permeable, the rock 105 may fracture at or near:

$${P}_{b} = \frac{3 {σ}_{min} - {σ}_{max} + T}{2 - β \frac{1 - 2 v}{1 - v}} , \qquad Equation ( 3 )$$

where β is Biot's coefficient and v is Poisson's ratio. Equation (3) is proposed by Haimson and Fairhurst and referred to as the H-F model. In the context of this disclosure, Equation (3) may be the first model or a modified version of Equation (3) may be the first model. For example, the breakdown pressure P_{b} may be considered a relative pressure above the pore pressure Pₚ and, as such, the true breakdown pressure P_{b} may be

$${P}_{b} = \frac{3 {σ}_{min} - {σ}_{max} + T}{2 - β \frac{1 - 2 v}{1 - v}} + {P}_{p} .$$

The elastic deformation of the rock 105 may be modeled using an elastic model. For example, if the rock 105 experiences isotropic confinement (i.e., σₘᵢₙ=σₘₐₓ=P₀), the elastic model may take the form:

$${P}_{elas} = 2 G \frac{u}{{a}_{0}} + {P}_{0} , \qquad Equation ( 4 )$$

where G is the shear modulus of the rock 105. However, a person of ordinary skill in the art will appreciate that other elastic models may be used to model the elastic deformation of the rock 105 under isotropic confinement. Further, a person of ordinary skill in the art will appreciate that the rock 105 may experience anisotropic confinement (i.e., σₘᵢₙ≠σₘₐₓ) without departing from the scope of the disclosure.

Returning to Equations (2) and (3), neither consider that the rock 105 may plastically deform following elastic deformation and prior to fracture. If the rock 105 plastically deforms prior to fracture, the rock 105 may actually fracture well above the breakdown pressure P_{b} determined using Equation (2) or (3) or neighboring rock may fracture instead. Hereinafter, the failure mode of the rock 105 following plastic deformation is denoted ductile fracture.

The yield pressure P_{y} at the yield point where the rock 105 transitions from elastic deformation to plastic deformation may be determined using the Mohr-Coulomb failure criterion where:

$${P}_{y} = \frac{UCS + {N}_{ϕ} ( 3 {σ}_{min} - {σ}_{max} )}{{N}_{ϕ} + 1} , \qquad Equation ( 5 )$$

where

$${N}_{ϕ} = \frac{1 + sin ϕ}{1 - sin ϕ} ,$$

ϕ is the friction angle, and UCS is the unconfined compressive strength. In the context of this disclosure, Equation (5) may be a second model or a modified version of Equation (5) may be the second model.

The plastic deformation of the rock 105 may be modeled using a plastic model. For example, if the rock 105 experiences isotropic confinement, the plastic model may take the form:

$${P}_{plas} = \frac{2 {N}_{ϕ} [ UCS + ( {N}_{ϕ} - 1 ) {P}_{0} ]}{( {N}_{ϕ} + 1 ) ( {N}_{ϕ} - 1 )} [ \frac{1 - {( \frac{{a}_{0}}{a} )}^{1 + \frac{1}{{N}_{ψ}}}}{1 - {( 1 - δ )}^{1 + \frac{1}{{N}_{ψ}}}} ] - \frac{UCS}{( {N}_{ϕ} - 1 )} , \qquad Equation ( 6 )$$

where

$${N}_{ψ} = \frac{1 + sin ψ}{1 - sin ψ} ,$$

ψ is dilation angle,

$$γ = \frac{{N}_{ϕ}}{{N}_{ψ}} \frac{{N}_{ψ} + 1}{{N}_{ϕ} - 1} , and δ = \frac{UCS + ( {N}_{ψ} + 1 ) {P}_{0}}{2 G ( {N}_{ϕ} - 1 )} .$$

Equation (6) may be evaluated analytically to determine Pₚₗₐₛ. However, a person of ordinary skill in the art will appreciate that other plastic models may be used to model the plastic deformation of the rock 105 under isotropic confinement. Further, a person of ordinary skill in the art will appreciate that the rock 105 may experience anisotropic confinement without departing from the scope of the disclosure. If the rock 105 experiences anisotropic confinement and both elastic deformation and plastic deformation, the elastic deformation and plastic deformation of the rock 105 may be numerically modeled using an elasto-plastic numerical model.

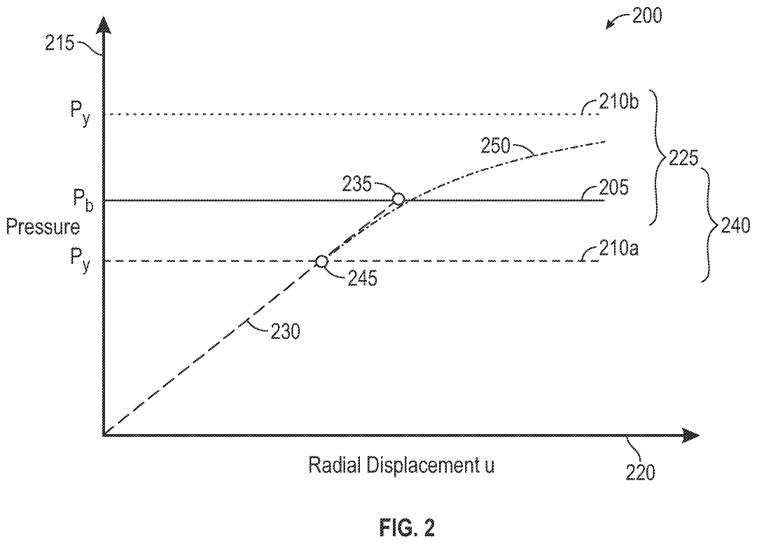

In some embodiments, the breakdown pressure P_{b} determined using Equation (2) or (3) and yield pressure P_{y} determined using Equation (5) may be compared to determine a deformation mechanism and failure mode of the rock 105. The deformation mechanism includes elastic deformation and, in some embodiments, plastic deformation. The failure mode includes brittle failure and ductile failure. Prior to brittle failure, the rock 105 experiences elastic deformation. Prior to ductile failure, the rock experiences elastic deformation followed by plastic deformation. For illustration, FIG. 2 displays a pressure-displacement curve 200 in accordance with one or more embodiments. The pressure-displacement curve 200 may be used to visually compare the breakdown pressure P_{b} 205 and yield pressure P_{y} 210a and 210b. Pressure is displayed along the ordinate 215. Radial displacement u 145 of the borehole 110 is displayed along the abscissa 220.

The breakdown pressure P_{b} 205 is displayed as a horizontal line in FIG. 2. Two values of the yield pressure P_{y} 210a and 210b are also displayed as horizontal lines in FIG. 2. The first yield pressure P_{y} 210a is displayed below the breakdown pressure P_{b} 205. The second yield pressure P_{y} 210b is displayed above the breakdown pressure P_{b} 205.

If the breakdown pressure P_{b} 205 is less than or below the yield pressure P_{y} 210b, illustrated as configuration I 225 in FIG. 2, the deformation mechanism and the failure mode of the rock 105 includes elastic deformation and brittle fracture, respectively. In some embodiments, the rock 105 elastically deforms based on the elastic model 230 of Equation (4) as illustrated in FIG. 2. If configuration I 225 occurs, the rock 105 fractures at or near the fracture point 235 where the breakdown pressure P_{b} 205 and elastic model 230 intersect.

If the breakdown pressure P_{b} 205 is greater than or above the yield pressure P_{y} 210a, illustrated as configuration II 240 in FIG. 2, the deformation mechanism and the failure mode of the rock 105 includes elastic deformation and plastic deformation and ductile fracture, respectively. In some embodiments, the rock 105 initially elastically deforms based on the elastic model 230 of Equation (4) and begins to plastically deform at the yield point 245 based on the plastic model 250 of Equation (6) as illustrated in FIG. 2. In these embodiments, the rock 105 begins to plastically deform at the yield point 245 where the yield pressure P_{y} 210a and elastic model 230 intersect. The rock 105 may plastically deform due to the rock 105 experiencing shear stresses. If configuration II 240 occurs, the rock 105 may fracture well above the breakdown pressure P_{b} 205 or neighboring rock may fracture instead.

The deformation mechanism, failure mode, and breakdown pressure P_{b} 205 may be used, at least in part, to design a completion plan for the borehole 110. The completion plan may be the plan designed to initially bring the borehole 110 into production or to return the borehole 110 to production.

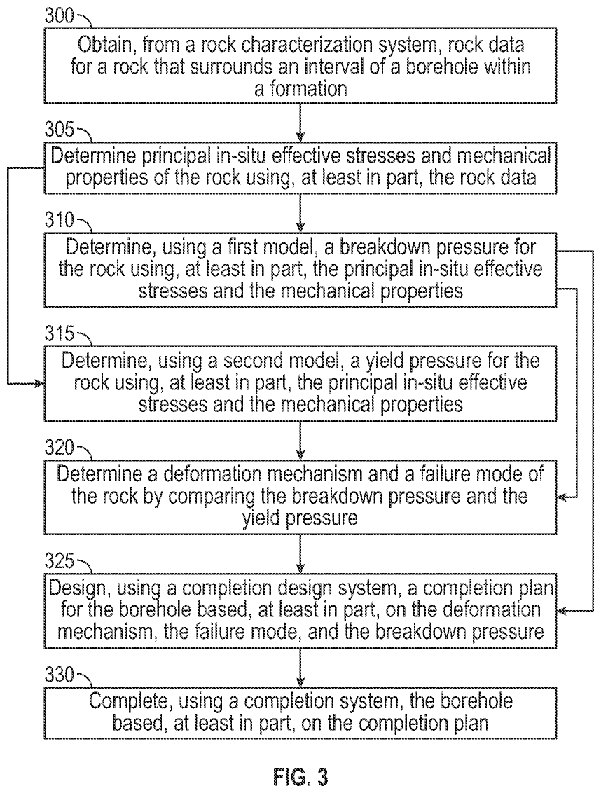

FIG. 3 describes a method of designing a completion plan for a borehole 110 in accordance with one or more embodiments.

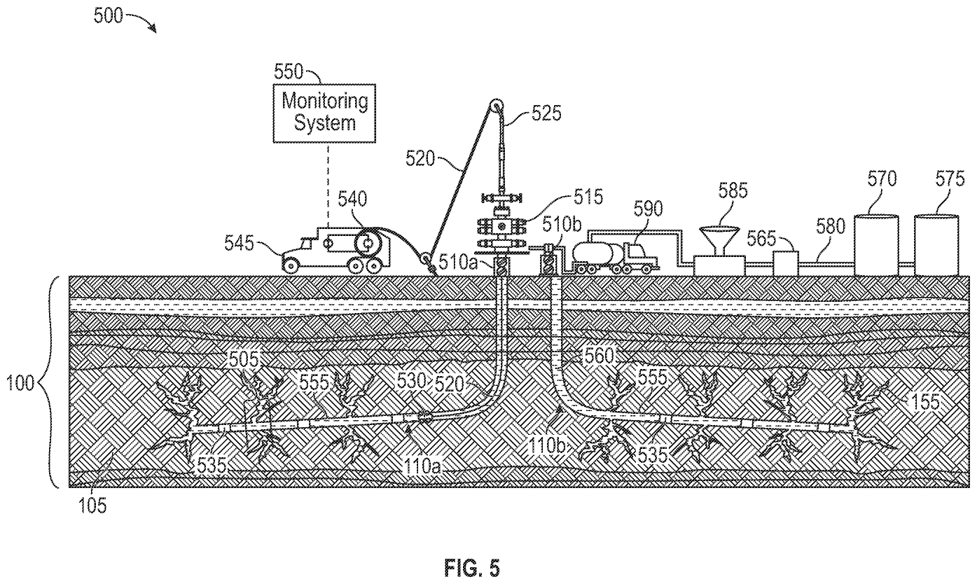

In step 300, rock data is obtained for a rock 105. The rock 105 surrounds an interval of a borehole 110 within a formation 100 as illustrated in FIG. 1. In some embodiments, the rock 105 is a part of a hydrocarbon reservoir within the formation 100 that the borehole 110 may penetrate. The rock data is obtained from a rock characterization system. The rock characterization system may include at least one of a well logging system, hydraulic fracturing system, and rock core characterization system. In some embodiments, the well logging system is configured to obtain a density log, acoustic/sonic log, and/or caliper log (hereinafter, the rock data or a portion of the rock data) downhole within the borehole 110. In other embodiments, the hydraulic fracturing system, as described in FIG. 5, is configured to perform a minifrac test and/or leakoff test to obtain the rock data or a portion of the rock data. In still other embodiments, the rock core characterization system is configured to obtain the rock data or a portion of the rock data from rock cores within a laboratory setting. The rock core characterization system may be configured to perform mechanical tests (e.g., tensile tests, compression tests, shear tests, and/or failure tests), tilt tests, porosity tests, permeability tests, etc. to obtain the rock data or a portion of the rock data.

In step 305, mechanical properties of the rock 105 are determined. The mechanical properties are determined directly or indirectly from the rock data obtained in step 300. In some embodiments, the mechanical properties include friction angle, tensile strength, and unconfined compressive strength. In other embodiments, the mechanical properties additionally include Biot's coefficient and Poisson's ratio.

In some embodiments, such as when the borehole 110 within the interval is vertical, the principal in-situ effective stresses may include the maximum horizontal principal in-situ effective stress σₘₐₓ 120 and minimum horizontal principal in-situ effective stress σₘᵢₙ 125 as illustrated in FIG. 1. In other embodiments, such as when the borehole 110 within the interval is lateral, the principal in-situ effective stresses may include the vertical principal in-situ effective stress and a horizontal principal in-situ effective stress. In some embodiments, the vertical principal in-situ effective stress may be determined by integration of a density log. In some embodiments, the maximum horizontal principal in-situ effective stress σₘₐₓ 120 and minimum horizontal principal in-situ effective stress σₘᵢₙ 125 may be determined from the rock data obtained from the minifrac test and/or leakoff test. In other embodiments, the maximum horizontal principal in-situ effective stress σₘₐₓ 120 and minimum horizontal principal in-situ effective stress σₘᵢₙ 125 may be determined from the sonic log. In still other embodiments, the maximum horizontal principal in-situ effective stress σₘₐₓ 120 may be inversely determined from the caliper log.

In some embodiments, the rock data determined from the tilt test may be used to determine the friction angle. In some embodiments, the tensile strength may be determined from the rock data obtained from the minifrac test or a tensile-to-failure test. In some embodiments, the unconfined compressive strength may be determined from the rock data obtained from the compressive test.

However, a person of ordinary skill in the art will appreciate that the above embodiments are not meant to be an exhaustive list of systems, tests, and methods used to determine the mechanical properties of the rock 105 but are offered as nonlimiting examples only. Any rock characterization system, whether used downhole or in a laboratory setting, any test, and any method may be used to determine the mechanical properties of the rock 105 without departing from the scope of the disclosure.

In step 310, the breakdown pressure P_{b} 205 is determined for the rock 105. To do so, a first model is relied on along with the principal in-situ effective stresses and tensile strength determined in step 305. If the borehole 110 is assumed to be impermeable, Equation (2) may be the first model. In these embodiments, the principal in-situ effective stresses σₘₐₓ and σₘᵢₙ and tensile strength T are input into the first model of Equation (2) to determine the breakdown pressure P_{b} 205. If the borehole 110 is assumed to be permeable, Equation (3) may be the first model. In these embodiments, the principal in-situ effective stresses σₘₐₓ and σₘᵢₙ, tensile strength T, Biot's coefficient, and Poisson's ratio are input into the first model of Equation (3) to determine the breakdown pressure P_{b} 205. However, a person of ordinary skill in the art will appreciate that the first model may be any model that may be used to determine the breakdown pressure P_{b} 205 of the rock 105 that assumes that the rock 105 elastically deforms.

In step 315, the yield pressure P_{y} 210a and 210b is determined for the rock 105. To do so, a second model is relied on along with the principal in-situ effective stresses, friction angle, and unconfined compressive strength determined in step 305. In some embodiments, Equation (5) may be the second model. In these embodiments, the principal in-situ effective stresses σₘₐₓ and σₘᵢₙ, friction angle, and unconfined compressive strength are input into the second model of Equation (5) to determine the yield pressure P_{y} 210a and 210b. However, a person of ordinary skill in the art will appreciate that the second model may be any model that determines the yield pressure P_{y} 210a and 210b of the rock 105.

In step 320, a deformation mechanism and failure mode of the rock 105 are determined. The deformation mechanism and failure mode are determined by comparing the breakdown pressure P_{b} 205 determined in step 310 and the yield pressure P_{y} 210a and 210b determined in step 315. If the breakdown pressure P_{b} 205 is less than or below the yield pressure P_{y} 210b, illustrated as configuration I 225 in FIG. 2, the deformation mechanism and failure mode of the rock 105 includes elastic deformation and brittle failure, respectively. If configuration I 225 occurs, the rock 105 fractures at or near the breakdown pressure P_{b} 205 at the fracture point 235 following elastic deformation. If the breakdown pressure P_{b} 205 is greater than or above the yield pressure P_{y} 210a, illustrated as configuration II 240 in FIG. 2, the deformation mechanism and failure mode of the rock 105 includes elastic deformation followed by plastic deformation and ductile failure, respectively. If configuration II 240 occurs, the rock 105 may fracture well above the breakdown pressure P_{b} 205 or neighboring rock may fracture instead following plastic deformation.

In step 325, a completion plan is designed for the borehole 110. The completion plan may be designed based, at least in part, on the deformation mechanism and failure mode determined in step 320 and the breakdown pressure P_{b} 205 determined in step 310. Details regarding what may be included within the completion plan are described below following a discussion of completion operations and completion systems.

If configuration I 225 occurs, designing the completion plan may include designing a matrix stimulation operation below the breakdown pressure P_{b} 205 and/or designing a hydraulic fracturing operation above the breakdown pressure P_{b} 205.

If configuration II 240 occurs, designing the completion plan may include generating a finite element model of the rock 105 that surrounds the interval of the borehole 110 within the formation 100. The finite element model may model or simulate the stress state (i.e., stress field) of the rock 105 over time as fluid is injected into the rock 105 via the borehole 110. In some embodiments, the finite element model may take the form of a finite element model, finite difference model, or finite volume model. The finite element model may include the elastic model 230, plastic model 250, and yield pressure Yₚ 210a and 210b as well as other structural/geometrical, mechanical, boundary condition, and/or mesh information. A person of ordinary skill in the art will appreciate that the combination of the elastic model 230 and plastic model 250 may be referred to as an elastoplastic model. In some embodiments, a finite element modeling system may be configured to generate the finite element model. The finite element modeling system may take the form of software located on a memory of a computer system.

In some embodiments, the finite element model may be used to determine an updated breakdown pressure P_{b} that reflects the true breakdown pressure P_{b} that causes the rock 105 to fracture following plastic deformation. In these embodiments, designing the completion plan may include designing a matrix stimulation operation below the updated breakdown pressure P_{b} and/or designing a hydraulic fracturing operation above the updated breakdown pressure P_{b}.

In other embodiments, the finite element model may be used to determine that the rock 105 does not fracture at a breakdown pressure P_{b} that can reasonably be applied to the rock 105. In these embodiments, designing the completion plan may include designing a matrix stimulation operation or not completing the borehole 110 along the interval. In these embodiments, designing the completion plan may exclude designing a hydraulic fracturing operation as the finite element model has determined that the rock 105 may not fracture at a breakdown pressure P_{b} that can reasonably be applied to the rock 105.

In still other embodiments, the finite element model may determine that surrounding rock fractures instead. In these embodiments, designing the completion plan may include designing a matrix stimulation operation below the updated breakdown pressure P_{b} for the surrounding rock and/or designing a hydraulic fracturing operation above the updated breakdown pressure P_{b} for the surrounding rock.

In some embodiments, a completion design system may be configured to design the completion plan. The completion design system may take the form of software located on the memory of the computer system.

In step 330, in some embodiments, the borehole 110 may be completed based, at least in part, on the completion plan designed in step 325. The borehole 110 may be completed using a completion system. If the completion plan includes a matrix stimulation operation, the completion system may include a matrix stimulation system. If the completion plan includes a hydraulic fracturing operation, the completion system may include a hydraulic fracturing system.

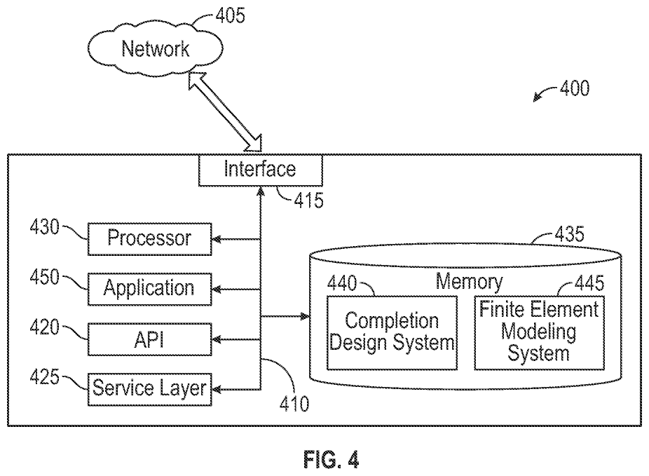

FIG. 4 illustrates a computer system 400 in accordance with one or more embodiments. The computer system 400 may be used to provide computational functionalities associated with described algorithms, methods, functions, processes, flows, and procedures as described in this disclosure, according to one or more embodiments. The illustrated computer system 400 is intended to encompass any computing device such as a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer system 400 may include a computer that includes an input device, such as a keypad, keyboard, touch screen, or other device that can accept user information, and an output device that conveys information associated with the operation of the computer system 400, including digital data, visual, or audio information (or a combination of information), or a GUI.

The computer system 400 can serve in a role as a client, network component, a server, a database or other persistency, or any other component (or a combination of roles) of a computer system for performing the subject matter described in the instant disclosure. The illustrated computer system 400 is communicably coupled with a network 405. In some implementations, one or more components of the computer system 400 may be configured to operate within environments, including cloud-computing-based, local, global, or other environment (or a combination of environments).

At a high level, the computer system 400 is an electronic computing device operable to receive, transmit, process, store, or manage data and information associated with the described subject matter. According to some implementations, the computer system 400 may also include or be communicably coupled with an application server, e-mail server, web server, caching server, streaming data server, business intelligence (BI) server, or other server (or a combination of servers).

The computer system 400 can receive requests over network 405 from a client application (for example, executing on another computer system 400) and responding to the received requests by processing the said requests in an appropriate software application. In addition, requests may also be sent to the computer system 400 from internal users (for example, from a command console or by other appropriate access method), external or third-parties, other automated applications, as well as any other appropriate entities, individuals, systems, or computer systems.

Each of the components of the computer system 400 can communicate using a system bus 410. In some implementations, any or all of the components of the computer system 400, both hardware or software (or a combination of hardware and software), may interface with each other or the interface 415 (or a combination of both) over the system bus 410 using an application programming interface (API) 420 or a service layer 425 (or a combination of the API 420 and service layer 425. The API 420 may include specifications for routines, data structures, and object classes. The API 420 may be either computer-language independent or dependent and refer to a complete interface, a single function, or even a set of APIs. The service layer 425 provides software services to the computer system 400 or other components (whether or not illustrated) that are communicably coupled to the computer system 400. The functionality of the computer system 400 may be accessible for all service consumers using this service layer. Software services, such as those provided by the service layer 425, provide reusable, defined business functionalities through a defined interface. For example, the interface may be software written in JAVA, C++, or other suitable language providing data in extensible markup language (XML) format or another suitable format. While illustrated as an integrated component of the computer system 400, alternative implementations may illustrate the API 420 or the service layer 425 as stand-alone components in relation to other components of the computer system 400 or other components (whether or not illustrated) that are communicably coupled to the computer system 400. Moreover, any or all parts of the API 420 or the service layer 425 may be implemented as child or sub-modules of another software module, enterprise application, or hardware module without departing from the scope of this disclosure.

The computer system 400 includes an interface 415. Although illustrated as a single interface 415 in FIG. 4, two or more interfaces 415 may be used according to particular needs, desires, or particular implementations of the computer system 400. The interface 415 is used by the computer system 400 for communicating with other systems in a distributed environment that are connected to the network 405. Generally, the interface 415 includes logic encoded in software or hardware (or a combination of software and hardware) and operable to communicate with the network 405. More specifically, the interface 415 may include software supporting one or more communication protocols associated with communications such that the network 405 or interface's hardware is operable to communicate physical signals within and outside of the illustrated computer system 400.

The computer system 400 includes at least one computer processor 430. Although illustrated as a single computer processor 430 in FIG. 4, two or more processors may be used according to particular needs, desires, or particular implementations of the computer system 400. Generally, the computer processor 430 executes instructions and manipulates data to perform the operations of the computer system 400 and any algorithms, methods, functions, processes, flows, and procedures as described in the instant disclosure.

The computer system 400 also includes a memory 435 that stores software and data. In some embodiments, the memory 435 may store a completion design system 440 that may be configured to perform step 325 as previously described relative to FIG. 3. In some embodiments, the memory 435 may store a finite element modeling system 445. Although illustrated as a single memory 435 in FIG. 4, two or more memories 435 may be used according to particular needs, desires, or particular implementations of the computer system 400 and the described functionality. While memory 435 is illustrated as an integral component of the computer system 400, in alternative implementations, memory 435 can be external to the computer system 400.

The application 450 is an algorithmic software engine providing functionality according to particular needs, desires, or particular implementations of the computer system 400, particularly with respect to functionality described in this disclosure. For example, application 450 can serve as one or more components, modules, applications, etc. Further, although illustrated as a single application 450, the application 450 may be implemented as multiple applications 450 on the computer system 400. In addition, although illustrated as integral to the computer system 400, in alternative implementations, the application 450 can be external to the computer system 400.

There may be any number of computer systems 400 associated with, or external to, a computer system containing a computer system 400, wherein each computer system 400 communicates over network 405. Further, the term “client,” “user,” and other appropriate terminology may be used interchangeably as appropriate without departing from the scope of this disclosure. Moreover, this disclosure contemplates that many users may use one computer system 400, or that one user may use multiple computer systems 400.

Following the design of the completion plan using the completion design system 440, the borehole 110 may be completed based on the completion plan. In some embodiments, completing the borehole 110 may include performing a matrix simulation operation within the rock 105 surrounding the interval of the borehole 110 or surrounding rock. In other embodiments, completing the borehole 110 may additionally or alternatively include performing a hydraulic fracturing operation within the rock 105 surrounding the interval of the borehole 110 or surrounding rock. In still other embodiments, completing the borehole 110 may include not completing the rock 105 surrounding the interval of the borehole 110.

The matrix stimulation operation (hereinafter “matrix stimulation”) is also known as a matrix treatment or matrix acidizing. Matrix stimulation is a treatment to increase the permeability of rock 105 within a hydrocarbon reservoir and, in turn, increase production. A matrix stimulation system may be configured to perform matrix stimulation. During matrix stimulation, a treatment fluid may be injected into the rock 105 via the borehole 110 at pressures below the breakdown pressure P_{b} 205 or updated breakdown pressure P_{b}. The treatment fluid may include, but is not limited to, an acid (e.g., hydrochloric acid, formic acid, and acetic acid), solvent, or other chemical.

If the hydrocarbon reservoir includes sandstone, matrix stimulation may restore or increase the permeability of the rock 105 within the formation 100 by removing debris from the formation 100, dissolving other material plugging the pores of the rock 105, or enlarging the pores of the rock 105. As such, information regarding the extent, type of formation debris, location, origin, reservoir mineralogy, and compatibility of the treatment fluid within the formation 100 may be additionally considered to design a completion plan that includes a matrix stimulation operation. To perform a matrix simulation operation in sandstone, for example, rock 105 may be preflushed with a hydrochloric acid, treated with a main treating fluid, and overflushed with a weak acid solution or brine. The main treating fluid is maintained under pressure inside the hydrocarbon reservoir for a period of time, after which the borehole 110 is swabbed and enters or is returned to production.

If the hydrocarbon reservoir includes carbonate, matrix stimulation generates new, highly conductive channels (i.e., wormholes) that bypass debris within the formation 100. As such, information regarding the temperature of the hydrocarbon reservoir, pumping rate, and fluid type may be additionally considered to design a completion plan that includes a matrix stimulation operation as each may affect the reactivity of the main treating fluid with the rock 105.

As such, if matrix stimulation is performed, the completion plan may include the goals of the completion plan, the fluid used for each of the preflush, treatment, and overflush, the location within the rock 105 that matrix stimulation will be performed on, the pressure of the main treating fluid downhole, and how long the main treating fluid will be downhole.

The hydraulic fracturing operation (hereinafter also “hydraulic fracturing”) is also known as “hydraulic stimulation” or colloquially “frac job.” FIG. 5 illustrates a hydraulic fracturing operation in accordance with one or more embodiments. The hydraulic fracturing operation and hydraulic fracturing system 500 are for illustration purposes only. The scope of the disclosure is intended to encompass any type of hydraulic fracturing operation and hydraulic fracturing system 500. In some embodiments, the completion design system 440 may transfer information associated with the completion plan to the hydraulic fracturing system 500 via the network 405 such that the hydraulic fracturing system 500 may perform the hydraulic fracturing operation at the location of the interval 505.

In some embodiments, the hydraulic fracturing operation is performed by separating the borehole 110 into multiple packed borehole lengths and fracturing each interval 505 in “stages.” Further, the hydraulic fracturing operation may be performed on multiple boreholes 110 that are geographically grouped. A single borehole 110 may have anywhere from one to more than forty stages. Typically, each stage includes one perforation operation and one pumping operation. While one operation is occurring on one borehole 110, a second operation may be performed on the other borehole 110. As such, FIG. 5 shows a hydraulic fracturing operation occurring on a first borehole 110a and a second borehole 110b. The first borehole 110a is undergoing the perforation operation and the second borehole 110b is undergoing the pumping operation.

The boreholes 110a and 110b are horizontal in that each borehole 110a and 110b includes a vertical section and a lateral section. The lateral section is a section of the borehole 110a and 110b that is drilled at least eighty degrees from vertical. The first borehole 110a is capped by a first frac tree 510a and the second borehole 110b is capped by a second frac tree 510b. Those of ordinary skill in the art will appreciate that the use of the term “frac” refers to “fracturing” and is used herein to describe elements that may be used in a hydraulic fracturing operation. Each frac tree 510a and 510b is similar to a Christmas/production tree but is specifically installed for the hydraulic fracturing operation. The frac trees 510a and 510b tend to have larger bores and higher-pressure ratings than a Christmas/production tree would have. Further, hydraulic fracturing operations require abrasive materials being pumped into the boreholes 110a and 110b at high pressures, so each frac tree 510a and 510b is designed to handle a higher rate of erosion.

In some embodiments, each borehole 110a and 110b requires four stages. Both the first borehole 110a and the second borehole 110b have undergone three stages and are undergoing the fourth stage. The second borehole 110b has already undergone the fourth stage perforation operation and is currently undergoing the fourth stage pumping operation. The first borehole 110a is undergoing the fourth stage perforating operation and has yet to undergo the fourth stage pumping operation.

In some embodiments, the perforating operation includes installing a wireline blow out preventor (BOP) 515 onto the first frac tree 510a. A wireline BOP 515 is similar to a drilling BOP. However, a wireline BOP 515 has seals designed to close around (or shear) wireline 520 rather than drill pipe. A lubricator 525 is connected to the opposite end of the wireline BOP 515. A lubricator 525 is a long, high-pressure pipe used to equalize between downhole pressure and atmosphere pressure to run downhole tools, such as a perforating gun 530, into the first borehole 110a.

The perforating gun 530 is pumped into the first borehole 110a using the lubricator 525, wireline 520, and fluid pressure. In accordance with one or more embodiments, the perforating gun 530 is equipped with explosives and a frac plug 535 prior to being deployed in the first borehole 110a. The wireline 520 is connected to a spool 540 often located on a wireline truck 545. Electronics (not pictured) included in the wireline truck 545 are used to control the unspooling/spooling of the wireline 520 and are used to send and receive messages along the wireline 520. The electronics may also be connected, wired or wirelessly, to a monitoring system 550 that is used to monitor and control the various operations being performed by the hydraulic fracturing system 500.

When the perforating gun 530 reaches a predetermined depth, a message is sent along the wireline 520 to set the frac plug 535. After the frac plug 535 is set, another message is sent through the wireline 520 to detonate the explosives, as shown in FIG. 5. The explosives create perforations in the casing 555 and in the surrounding formation 100. There may be more than one set of explosives on a singular perforating gun 530, each detonated by a distinct message. Multiple sets of explosives are used to perforate different depths along the casing 555 for a singular stage. Further, the frac plug 535 may be set separately from the perforation operation without departing from the scope of the disclosure herein.

As explained above, FIG. 5 shows the second borehole 110b undergoing the pumping operation after the fourth stage perforating operation has already been performed and perforations are left behind in the casing 555 and the surrounding formation 100. A pumping operation includes pumping a frac fluid 560 into the perforations to propagate the perforations and create fractures 155 in the surrounding formation 100. The frac fluid 560 often includes a certain percentage of water, proppant, and chemicals.

FIG. 5 further shows chemical storage containers 565, water storage containers 570, and proppant storage containers 575 that are constituents of the hydraulic fracturing system 500. Frac lines 580 and transport belts (not pictured) transport the chemicals, proppant, and water from the storage containers 565, 570, 575 into a frac blender 585. Sensors (not pictured) are located throughout this equipment to send signals to the monitoring system 550. The monitoring system 550 may be used to control the volume of water, chemicals, and proppant used in the pumping operation.

The frac blender 585 blends the water, chemicals, and proppant to become the frac fluid 560. The frac fluid 560 is transported to one or more frac pumps, often pump trucks 590, to be pumped through the second frac tree 510b into the second borehole 110b. Each pump truck 590 includes a pump designed to pump the frac fluid 560 at a certain pressure. More than one pump truck 590 may be used at a time to increase the pressure of the frac fluid 560 being pumped into the second borehole 110b. The frac fluid 560 is transported from the pump truck 590 to the second frac tree 510b using frac lines 580.

The fluid pressure propagates and creates the fractures 155 while the proppant props open the fractures 155 once the pressure is released. Different chemicals may be used to lower friction pressure, prevent corrosion, etc. The pumping operation may be designed to last a certain length of time to ensure the fractures 155 have sufficiently propagated. Further, the frac fluid 560 may have different make ups throughout the pumping operation to optimize the pumping operation without departing from the scope of the disclosure herein.

When the hydraulic fracturing operation is completed on either borehole 110a and 110b, the frac trees 510a and 510b must be removed from each borehole 110a and 110b to perform the final completion operations which include drilling out the plugs 535 using coiled tubing or a snubbing unit and installing production tubing (not pictured). The production tubing is installed by running the length of production tubing into each borehole 110a and 110b and landing out the tubing hanger (i.e., the surface extending portion of the production tubing that has seals) into a tubing head that caps each borehole 110a and 110b.

As such, if hydraulic fracturing is performed, the completion plan may include the number of stages that will be performed on the borehole 110, the length of each interval 505, when, where, and for how long each perforation operation and pumping operation will be performed, what and when frac fluids 560 and proppant are pumped into the borehole 110, what sets of explosives are used for each perforation operation, and what final completion operations will be used.

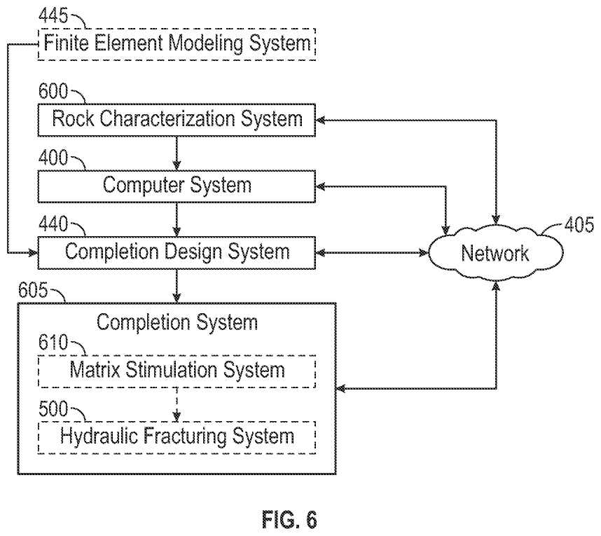

FIG. 6 illustrates a flowchart of systems in accordance with one or more embodiments. Systems within a dashed-line box may be optional. The rock characterization system 600 may be configured to obtain the rock data from the rock 105. The rock characterization system 600 may include at least one of a well logging system, hydraulic fracturing system 500, and rock core characterization system. The rock data may be transferred to and stored on the computer system 400 via the network 405. The computer system 400 may be configured to perform steps 300, 305, 310, 315, and 320. A completion design system 440 may be configured to perform step 325. In some embodiments, a finite element modeling system 445 may be configured to design the completion plan. For example, the finite element modeling system 445 may be configured to generate the finite element model and determine an updated breakdown pressure if configuration II 240 occurs.

Following the design of the completion plan, the completion plan may be transferred to and stored on the completion system 605 via the network 405. The completion system 605 may be configured to complete the borehole 110 based, at least in part, on the completion plan. If configuration I 225 occurs, the completion system 605 may include a matrix stimulation system 610 and/or hydraulic fracturing system 500. The matrix stimulation system 610 may be configured to perform a matrix stimulation operation on the rock 105 surrounding the interval 505 of the borehole 110 or surrounding rock. The hydraulic fracturing system 500 may be configured to perform a hydraulic fracturing operation on the rock 105 surrounding the interval 505 of the borehole 110 or surrounding rock as described relative to FIG. 5. However, if the finite element modeling system 445 determines that the rock 105 does not fracture at a breakdown pressure P_{b} that can reasonably be applied to the rock 105, the completion system 605 may not include the hydraulic fracturing system 500 and a hydraulic fracturing operation is not performed.

### EXAMPLES

Hereinafter, an example is provided to demonstrate the disclosed method as described in the Detailed Description section.

Assume rock 105 surrounds an interval 505 of a borehole 110, the borehole 110 is impermeable, isotropic confining stress σ_{c} is 22.5 megapascals (MPa), pore pressure pₚ is 10 MPa, tensile strength T is 5 MPa, Young's modulus E is 1000 MPa, Poisson's ratio v is 0.2, cohesion strength c is 5 MPa, friction angle ϕ is 30°, dilation angle ψ is 10°, Biot's coefficient β is 1, and initial radius a₀ is 0.1 meters (m).

The maximum horizontal principal in-situ effective stress σₘₐₓ 120 and minimum horizontal principal in-situ effective stress σₘᵢₙ 125 may be determined where:

$${P}_{0} = {σ}_{max} = {σ}_{min} = {σ}_{c} - β {p}_{p} = 22.5 - 1 \cdot 10 = 12.5 MPa .$$

The breakdown pressure P_{b} 205 is determined using Equation (2) as the first model where:

$${P}_{b} = 3 {σ}_{min} - {σ}_{max} + T + {P}_{p} = 3 \cdot 12.5 - 12.5 + 5 + 10 = 40 MPa .$$

The yield pressure P_{y} 210a and 210b is determined using Equation (5) as the second model where:

$$UCS = \frac{2 c cos ϕ}{1 - sin ϕ} = \frac{2 \cdot 5 cos 30 °}{1 - sin 30 °} = 17.3 MPa ,$$

$${N}_{ϕ} = \frac{1 + sin ϕ}{1 - sin ϕ} = \frac{1 + sin 30 °}{1 - sin 30 °} = 3 , and$$

$${P}_{y} = \frac{UCS + {N}_{ϕ} ( 3 {σ}_{min} - {σ}_{max} )}{{N}_{ϕ} + 1} = \frac{17.32 + 3 ( 3 \cdot 12.5 - 12.5 )}{3 + 1} = 23.1 MPa .$$

The elastic model is determined by assuming linear homogenous isotropic elasticity where:

$$G = \frac{E}{2 ( 1 + v )} = \frac{1000}{2 ( 1 + 0.2 )} = 416.7 MPa ,$$

and plotting Pₑₗₐₛ relative to

$$\frac{a}{{a}_{0}}$$

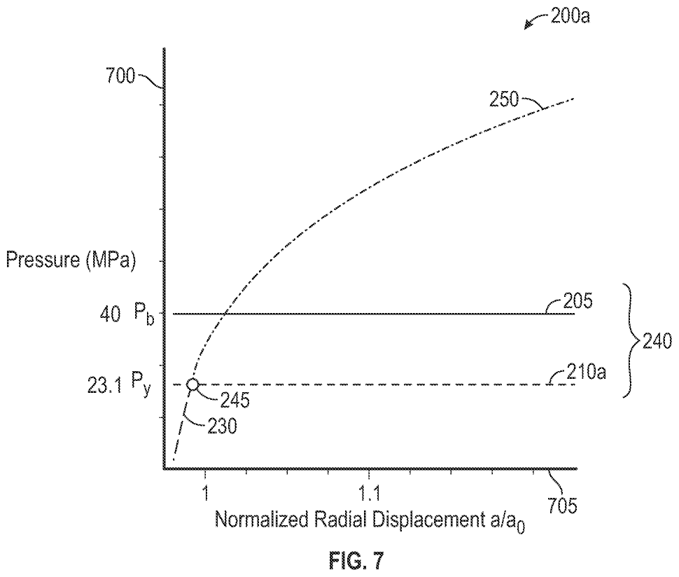

using Equation (4) as uisprayeu as the elastic model 230 in FIG. 7.

The plastic model is determined by plotting Pₚₗₐₛ relative to

$$\frac{a}{{a}_{0}}$$

using Equation (6) as displayed as the plastic model 250 in FIG. 7.

FIG. 7 displays an example pressure-displacement curve 200a in accordance with one or more embodiments. The example pressure-displacement curve 200a may be used to visually compare the breakdown pressure P_{b} 205 and yield pressure P_{y} 210a. Pressure is displayed along the ordinate 700. Normalized radial displacement a/a₀ of the borehole 110 is displayed along the abscissa 705.

Comparing the breakdown pressure P_{b} 205 of 30 MPa to the yield pressure P_{y} 210a of 23.1 MPa, the breakdown pressure P_{b} 205 is greater than the yield pressure P_{y} 210a (i.e., configuration II 240 occurs). Thus, the deformation mechanism and failure mode of the rock 105 includes elastic deformation followed by plastic deformation and ductile fracture, respectively. In some embodiments, as illustrated in FIG. 7, the rock 105 may elastically deform based on the elastic model 230 of Equation (4), yield at the yield point 245, and then plastically deform based on the plastic model 250 of Equation (6).

As such, the finite element modeling system 445 may be configured to determine an updated breakdown pressure P_{b}. A matrix stimulation operation may be performed below the updated breakdown pressure P_{b} and/or a hydraulic fracture operation may be performed above the updated breakdown pressure P_{b} or the borehole 110 along the interval 505 may not be completed.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed is:

1. A method comprising:
obtaining, from a rock characterization system, rock data for a rock that surrounds an interval of a borehole within a formation;
determining principal in-situ effective stresses and mechanical properties of the rock using, at least in part, the rock data;
determining, using a first model, a breakdown pressure for the rock using, at least in part, the principal in-situ effective stresses and the mechanical properties;
determining, using a second model, a yield pressure for the rock using, at least in part, the principal in-situ effective stresses and the mechanical properties;
determining a deformation mechanism and a failure mode of the rock by comparing the breakdown pressure and the yield pressure; and
designing, using a completion design system, a completion plan for the borehole based, at least in part, on the deformation mechanism, the failure mode, and the breakdown pressure.

2. The method of claim 1, wherein the mechanical properties comprise a tensile strength, and
wherein determining the breakdown pressure further comprises using the tensile strength.

3. The method of claim 2, wherein the mechanical properties further comprise a Biot's coefficient and a Poisson's ratio, and
wherein determining the breakdown pressure further comprises using the Biot's coefficient and the Poisson's ratio.

4. The method of claim 1, wherein the mechanical properties comprise a friction angle and an unconfined compressive strength, and
wherein determining the yield pressure further comprises using the friction angle and the unconfined compressive strength.

5. The method of claim 1, further comprising:
using a finite element modeling system:
generating a finite element model of the formation based, at least in part, on the yield pressure, and
determining an updated breakdown pressure of the rock based, at least in part, on the finite element model.

6. The method of claim 5, wherein the finite element model comprises an elastic model and a plastic model for the rock.

7. The method of claim 1, further comprising completing, using a completion system, the borehole based, at least in part, on the completion plan.

8. The method of claim 7, wherein completing the borehole comprises performing, using a hydraulic fracturing system, a hydraulic fracturing operation on the rock surrounding the interval of the borehole.

9. The method of claim 1, wherein the second model comprises a Mohr-Coulomb failure criterion.

10. The method of claim 1, wherein the deformation mechanism comprises elastic deformation and the failure mode comprises brittle fracture when the breakdown pressure is less than the yield pressure, and
wherein the deformation mechanism comprises plastic deformation and the failure mode comprises ductile fracture when the breakdown pressure is greater than the yield pressure.

11. A system comprising:
a computer system configured to:
receive, from a rock characterization system, rock data for a rock that surrounds an interval of a borehole within a formation,
determine principal in-situ effective stresses and mechanical properties of the rock using, at least in part, the rock data,
determine, using a first model, a breakdown pressure for the rock using, at least in part, the principal in-situ effective stresses and the mechanical properties,
determine, using a second model, a yield pressure for the rock using, at least in part, the principal in-situ effective stresses and the mechanical properties, and
determine a deformation mechanism and a failure mode of the rock by comparing the breakdown pressure and the yield pressure; and
a completion design system configured to design a completion plan for the borehole based, at least in part, on the deformation mechanism, the failure mode, and the breakdown pressure.

12. The system of claim 11, further comprising the rock characterization system configured to obtain the rock data.

13. The system of claim 12, wherein the rock characterization system comprises at least one of a well logging system, a hydraulic fracturing system, and a rock core characterization system.

14. The system of claim 11, further comprising a finite element modeling system configured to:
generate a finite element model of the formation based, at least in part, on the yield pressure; and
determine an updated breakdown pressure of the rock based, at least in part, on the finite element model.

15. The system of claim 11, further comprising a completion system configured to complete the borehole based, at least in part, on the completion plan.

16. The system of claim 15, wherein the completion system comprises a hydraulic fracturing system configured to perform a hydraulic fracturing operation on the rock surrounding the interval of the borehole.

17. The system of claim 11, wherein the deformation mechanism comprises elastic deformation and the failure mode comprises brittle fracture when the breakdown pressure is less than the yield pressure, and
wherein the deformation mechanism comprises plastic deformation and the failure mode comprises ductile fracture when the breakdown pressure is greater than the yield pressure.

18. A non-transitory computer-readable memory having computer-executable instructions stored thereon that, when executed by a computer processor, perform steps comprising:
receiving, from a rock characterization system, rock data for a rock that surrounds an interval of a borehole within a formation;
determining principal in-situ effective stresses and mechanical properties of the rock using, at least in part, the rock data;
determining, using a first model, a breakdown pressure for the rock using, at least in part, the principal in-situ effective stresses and the mechanical properties;
determining, using a second model, a yield pressure for the rock using, at least in part, the principal in-situ effective stresses and the mechanical properties;
determining a deformation mechanism and a failure mode of the rock by comparing the breakdown pressure and the yield pressure; and
designing, using a completion design system, a completion plan for the borehole based, at least in part, on the deformation mechanism, the failure mode, and the breakdown pressure.

19. The non-transitory computer-readable memory of claim 18, wherein the steps further comprise:
using a finite element modeling system:
generating a finite element model of the formation based, at least in part, on the yield pressure, and
determining an updated breakdown pressure of the rock based, at least in part, on the finite element model.

20. The non-transitory computer-readable memory of claim 18, wherein the deformation mechanism comprises elastic deformation and the failure mode comprises brittle fracture when the breakdown pressure is less than the yield pressure, and
wherein the deformation mechanism comprises plastic deformation and the failure mode comprises ductile fracture when the breakdown pressure is greater than the yield pressure.
